(19) United States
(12) Patent Application Publication
(10) Pub. No.: US 2026/0266755 A1
(43) Pub. Date: Sep. 10, 2026

(54) THREE-DIMENSIONAL BIOSENSORS AND METHOD OF MANUFACTURING USING SUBSTRATE STRUCTURE

(71) Applicant: Kavin Krishna, Sugar Land, TX (US)

(72) Inventor: Kavin Krishna, Sugar Land, TX (US)

(21) Appl. No.: 19/048,863

(22) Filed: Feb. 8, 2025

Related U.S. Application Data
(60) Provisional application No. 63/569,195, filed on Mar. 24, 2024.

(51) Int. Cl. G01N 27/327; B01L 3/00; G01N 33/543

(52) U.S. Cl. CPC G01N 27/3273; B01L 3/502707; B01L 3/502715; G01N 33/543; B01L 2300/0645; B01L 2300/12

(57) ABSTRACT

Diverse approaches and apparatuses are associated with the creation of three-dimensional biosensors, as outlined and claimed in this documentation. In particular embodiments, a three-dimensional biosensor includes a structural core that enhances mechanical stability, thermal management, and electrical conductivity, expanding the effective surface area for biomolecular interactions beyond the biosensor's physical footprint. The biosensor consists of a core layer with a primary surface, from which an array of microstructured elements extends. Various selective layers are applied to the core for capturing specific analytes. This configuration hosts bio-recognition sites with active materials chosen for their affinity to target biomolecules. Certain embodiments pertain to fabrication processes, incorporating conductive and selective material deposition or integrating the backbone within the biosensor without additive layers, depending on functionality.

### BACKGROUND OF THE INVENTION

### Field of the Invention

This invention pertains to manufacturing techniques for three-dimensional biosensors that convert the detection of biological targets into measurable electrochemical signals.

### Summary of the Related Art

The literature suggests that increasing the surface area of biosensors through 3D structures could substantially improve these aspects by allowing more extensive interaction between the sensor's surface and the target analytes. U.S. Patent Application 20130206595 describes a biosensor with a three-dimensional structure created using 3D Molded Interconnect Device (MID) technology on a polymer substrate, where reaction electrodes and signal transfer parts are spatially formed to enhance sensitivity and connectivity. It aims to overcome the limitations of conventional planar biosensors by enabling versatile three-dimensional configurations, simplifying manufacturing processes, reducing costs, and improving performance through various 3D structuring techniques. Another U.S. Pat. No. 8,323,465 describes an invention focused on improving chemical sensors, particularly biosensors, through the development of a three-dimensional ordered macroporous (3DOM) sensor apparatus. This 3DOM sensor aims to address the limitations of existing microporous and mesoporous materials by offering a structure with larger, more uniform pores.

There is a value in making a three-dimensional biosensor device due to the increased rate of reaction despite a small geometric area. This energy could be in various forms, such as current or charge potential in a Potentiometric device. The benefits include higher sensitivity by increasing surface area for chemical reactions, increasing binding affinity, faster response times, and improved signal-to-noise ratio. However, there are significant issues that prevent the commercialization of these biosensors. One of the big problems is to ensure the mechanical stability of the bio-recognition elements in the three-dimensional biosensor. Another issue is maintaining consistent electrical conductivity throughout the various layers of a three-dimensional biosensor structure. The present invention provides a novel way of making three-dimensional electrochemical biosensors that not only enhance their analytical performance but also offer significant advantages in terms of weight and size while combating the above issues of 3D structures. The above-mentioned U.S. Patent Application 20130206595 emphasizes the steric formation of components to achieve three-dimensional structures. This differs from the proposed backbone substrate structure approach that emphasizes material variety and manufacturing techniques for enhancing surface area and functional properties.

### SUMMARY OF THE INVENTION

This patent outlines methods for the creation of three-dimensional (3D) biosensors with superior sensing abilities, leveraging an innovative structural core. This core structure is crucial for the biosensor's design, providing improved mechanical stability and electrical conductivity as needed. It notably increases the surface area in comparison to the bio sensor's physical size, enhancing the device's ability to detect and measure specific biomolecules effectively.

The core layer can be made from a wide array of materials, including but not limited to metals and ceramics, and is shaped in a manner that maximizes surface area—such shapes may include, but are not limited to, configurations like columns. This structure is then covered with a selective layer that is specific to the analyte of interest, allowing for precise identification and measurement. It should be highlighted that the choice and application of these selective layers are beyond the scope of this patent.

The structural design of the core can take on various forms that improve the ratio of surface area to physical size, encompassing designs such as two columns.

In certain embodiments, the foundational framework of the biosensor is crafted from an array of materials, including but not limited to alloys, compound semiconductors, polymers, bio-glasses, and crystalline substances, through various removal processes. When considering disposable biosensors, materials that naturally degrade over time, such as polylactic acid (PLA), polyglycolic acid (PGA), and their composites, are favored to ensure functionality throughout their usage period followed by environmentally friendly decomposition. These materials undergo precision shaping via methods such as laser ablation, which employs a focused laser beam to selectively remove material, or by photolithography coupled with wet chemical etching for intricate patterning. In addition, techniques like direct writing using conductive ink for circuitry or focused ion beam (FIB) milling offer alternatives for meticulously removing sections of material to achieve the desired biosensor architecture.

In some variations, the fabrication of the structural core leverages diverse additive manufacturing strategies, engaging a broad spectrum of materials. This process initiates with the creation of a provisional mold through advanced methods like 3D printing, which sets the stage for the core material's infusion. Subsequent stages may involve innovative techniques such as laser-induced forward transfer (LIFT), aerosol jet printing, or direct ink writing for material deposition. In particular situations, the foundational layer is directly formed through techniques like micro-contact printing or nano-imprint lithography. This layer could then be further structured on a flat substrate using state-of-the-art deposition processes, before being meticulously assembled with precision robotics and secured via methods beyond soldering, such as using conductive adhesives or ultrasonic welding, to achieve a robust and efficient biosensor architecture.

The active material can be directly coated onto and encompassing the conductive substance through several techniques, such as electrochemical plating, electroless plating, simultaneous deposition within organic or inorganic frameworks, electrophoretic plating, mechanical stuffing and compression, and deposition aided by vacuum flow.

Standard deposition techniques, such as sputter deposition, evaporation, electrophoretic deposition, vapor-assisted vacuum deposition (e.g., Atomic Layer Deposition (ALD) or Chemical Vapor Deposition (CVD), and electrochemical or electroless deposition, are first used to apply a conductive layer in scenarios involving an electrically non-conductive backbone. Using techniques like sputter etching, ion milling, and lift-off, this deposited conductive layer must then be selectively removed to cut off the electrical pathway between the anode and cathode backbones. Furthermore, to prevent unwanted removal of certain areas, lithography can be used in conjunction with chemical dissolution.

In addition, the backbone can embed microfluidic channels to facilitate the directed flow of samples over the sensor surface. This can enhance analyte-receptor interactions and improve the washout of non-bound entities, leading to faster and more sensitive detection.

This invention integrates a thermal regulation system within the biosensor framework, which is essential for stabilizing operational temperatures and ensuring consistent analytical performance. Heat dissipation is facilitated by integrating materials with high thermal conductivity yet low electrical conductivity, such as specific ceramics or composite materials, into the biosensor's foundational core. These materials are adept at serving as heat sinks while preserving the biosensor's electrical isolation.

The biosensor's architecture includes a network of thermally conductive channels that utilize advanced non-metallic, thermally conductive substances, such as boron nitride or silicon carbide, to transport heat efficiently from temperature-sensitive zones, thereby preventing hotspots and promoting a consistent thermal environment throughout the device.

In scenarios demanding superior thermal management, the biosensor adopts a stratified structure with interspersed layers of thermally conductive ceramics and thermal insulation. This arrangement mitigates heat flow and is particularly effective in high-power applications or under variable temperature conditions.

For instances where active heat expulsion is critical, the design features a passive cooling system inclusive of thermally conductive vias or through-holes that traverse the insulating layer (layer 1), providing a direct thermal pathway from hot regions to a heat-dissipating interface, thereby leveraging natural convective processes for enhanced thermal regulation.

The fabrication of the thermally conductive but electrically insulating through-holes begins with precision drilling into the biosensor's foundational layer to create a pathway for heat transfer. The material chosen for the through-holes is paramount, but one embodiment uses ceramics or polymer composites that exhibit high thermal conductivity and intrinsic electrical insulation.

Upon establishing the through-holes, they are filled with the selected thermally conductive material using a method tailored to the material properties and biosensor design. This could involve sintering for ceramic fills or a deposition process for polymer composites, ensuring a complete fill without voids.

Subsequent to filling, the surface of the biosensor is planarized to maintain a uniform surface topology, which is critical for sensor function and reliability. The planarization process, such as Chemical Mechanical Planarization (CMP), is carefully controlled to achieve a smooth finish without compromising the thermal integrity of the filled through-holes.

### BRIEF DESCRIPTION OF THE DRAWINGS

The figures provided herein illustrate the novel construction of three-dimensional biosensor using a specialized backbone layer and are intended solely for illustrative purposes, not to constrain the scope of the invention.

FIG. 1 illustrates a cross-sectional view of a three-dimensional biosensor with an alternating electrode pattern and an innovative copper thermal regulation layer with through-holes, featuring a backbone substrate structure for mechanical support and insulators to maintain electrical separation.

FIG. 2 is an expanded view of the biosensor's electrodes, with cylinders as negative electrodes and positive electrodes, indicating the layered composition of the active materials within the biosensor structure.

FIG. 3 depicts the manufacturing stages of the backbone substrate structure using subtractive reactive ion etching, where a photoresist masking layer patterns the backbone atop the through-hole copper layer, employing optical lithography to define the structure before etching.

### DETAILED DESCRIPTION OF THE INVENTION

The invention delineates an advanced methodology for constructing multi-dimensional biosensors, leveraging a structurally crucial backbone substrate layer. This inventive layer bestows upon the device enhanced mechanical stability and optimized thermal connectivity. It also significantly increases the effective surface area for electrochemical reactions compared to its two-dimensional geometric projections, contributing to improved device efficiency. An illustrative embodiment of this invention is the creation of a pillar-like backbone substrate structure. Following this, the resulting framework is coated with active materials, specifically cathode or anode substances, which are instrumental in the operational assembly of the biosensor. Various materials—encompassing metals, semiconductors, and ceramics—and sophisticated manufacturing techniques such as subtractive etching and additive deposition can be used here.

The envisaged three-dimensional biosensors are crafted to surpass the performance of standard flat sensors, especially in spatially restricted applications while maintaining a high functional density. Their three-dimensional configuration facilitates a more effective interaction between the sensor elements and target analytes, optimizing the sensor's responsiveness and selectivity in environments where compactness is essential, such as but not limited to wearable health monitors and dense lab-on-a-chip devices.

At the heart of the invention lies a robust, conductive backbone that acts as the underlying structure of the biosensor, although it remains passive in the biochemical interactions. It is designed to ensure not just mechanical durability but also to maintain consistent electrical properties across the sensor. This backbone maximizes the active surface area for the biosensor's selective layers, promoting enhanced durability and consistent performance across the biosensor's operational lifespan.

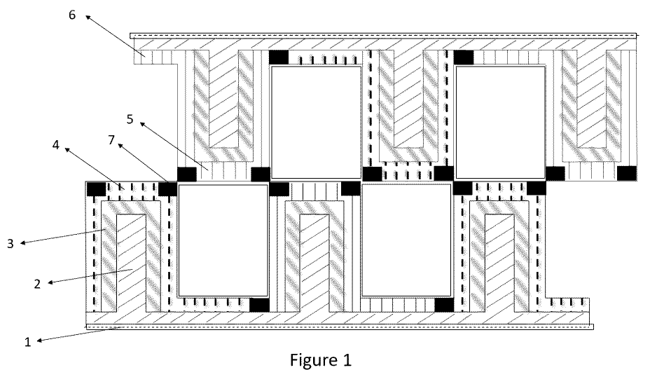

FIG. 1 depicts an intricate three-dimensional biosensor architecture, incorporating a copper base layer (1) designed with through-holes for adept thermal regulation. The biosensor utilizes a backbone substrate structure (2) which forms the primary framework, offering substantial support for the conductive material (3) that is crucial for electrical flow. This design is characterized by an inverted configuration of the conductive elements to maximize surface area for electron exchange, thereby enhancing rate of reaction. The negative electrodes (4) and the positive electrodes (5) are laid out in an alternating pattern, resembling a bricklaying motif that aids in optimizing the reactive surface area within the biosensor. To ensure the mechanical integrity and prevent electrical short-circuiting, insulators are employed between the positive and negative electrodes. Additionally, the top part of the structure (6) presents an inverted layout of the conductive material, contributing to the maximization of reactivity across the biosensor system. This sophisticated arrangement aims to provide a balance between high energy density, efficient thermal management, and increased reactivity.

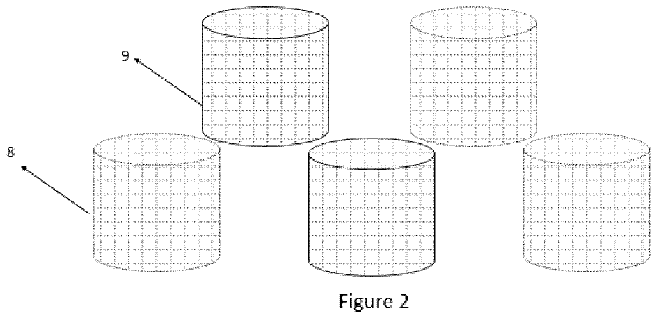

FIG. 2 provides a detailed representation of the electrode components utilized in the three-dimensional biosensor design. Shown are cylindrical structures, with item 8 representing the negative electrode and item 9 representing the positive electrode. Each cylinder is patterned with a grid that signifies the active material layers that contribute to the biosensor's electrochemical reactions. The distinct patterning implies varying material thickness or composition to optimize the electrode functions and enhance the biosensor's performance.

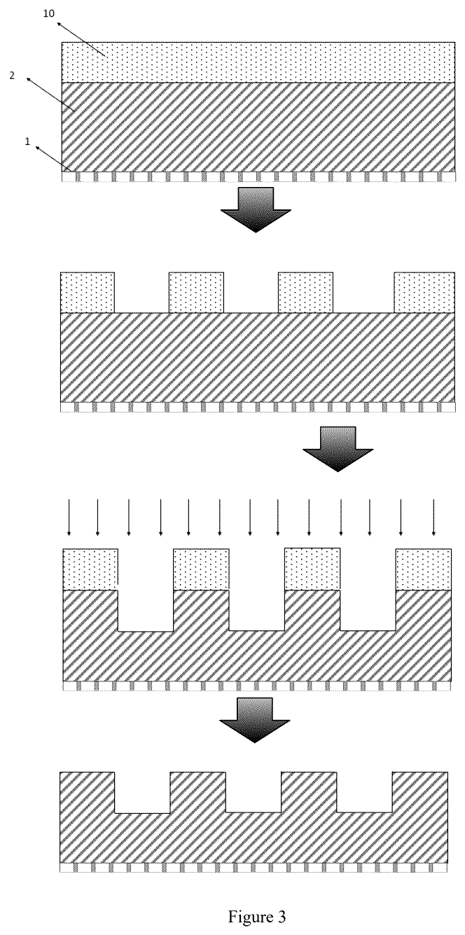

FIG. 3 illustrates the sequential steps in the subtractive manufacturing process of a three-dimensional biosensor's backbone substrate structure using reactive ion etching. The first panel shows a composite layer, where (1) is the through-hole copper layer for thermal management and (2) represents the initial backbone substrate structure. Atop the backbone, (10) indicates a photoresist layer applied to pattern the backbone substrate structure. The second panel reflects the application of optical lithography which transfers the desired pattern onto the photoresist layer (10). This pattern corresponds to the areas of the backbone that will remain after the etching process. The third panel shows the post-lithography state, where the exposed portions of the photoresist have been developed away, revealing the areas of the underlying backbone material (2) that will be etched. The final panel demonstrates the result after the reactive ion etching process has been completed. The areas unprotected by the photoresist have been removed, leaving a patterned backbone substrate structure. The etching creates a precise and consistent pattern that correlates with the desired geometry for the biosensor's electrodes and thermal regulation system. The remaining photoresist can then be cleared, leaving the patterned copper layer (1) and the newly formed backbone substrate structure (2).

### EXAMPLE 1

This example details the application of the backbone substrate structure in a biosensor designed for early detection of pathogens in a clinical setting. The biosensor utilizes a substrate of gold-patterned glass on which a conductive polymer backbone is constructed. The polymer, selected for its biocompatibility and electrical properties, is deposited onto the glass substrate using a screen-printing technique. A photoresist layer is applied over the polymer, and ultraviolet (UV) lithography is employed to create a precise pattern. The exposed areas of the polymer are then etched away using an oxygen plasma, which ensures high fidelity to the UV-defined pattern. The resulting structure forms the scaffold for subsequent immobilization of biological recognition elements specific to the pathogen of interest.

In the following stage, the biosensor surface is modified with self-assembled monolayers (SAMs) of thiol-functionalized compounds, which provide a reactive foundation for attaching the biological recognition molecules. The SAMs are deposited through microcontact printing, which precisely transfers the thiol compounds to the polymer backbone. Afterward, antibodies specific to a target pathogen are grafted onto the SAM-treated surface, creating a selective layer capable of capturing the pathogen from a sample matrix.

The biosensor is integrated into a microfluidic device, which channels the sample over the sensor surface. Interaction between the target pathogen and the immobilized antibodies results in a measurable change in the electrical properties of the conductive polymer, correlating with pathogen concentration. The biosensor's response is evaluated using an integrated circuit that quantifies changes in conductance, enabling real-time detection and monitoring of pathogen levels. This rapid and sensitive diagnostic tool has significant implications for patient care and the management of infectious diseases.

### EXAMPLE 2

This example illustrates the utilization of the novel biosensor architecture for the detection of glucose levels in bodily fluids, which is critical for managing diabetes. The biosensor is constructed on a silicon substrate where a nanostructured titanium dioxide (TiO2) backbone layer is fabricated using atomic layer deposition (ALD). This nanostructured backbone is designed to provide a high surface-to-volume ratio, which is key for enzyme immobilization and sensor sensitivity.

To pattern the TiO2 layer, a high-resolution photoresist is spin-coated onto the substrate and selectively exposed to UV light through a mask defining the sensor's architecture. The exposed photoresist is developed, and the TiO2 beneath the unexposed areas is removed using a wet etching solution, precisely shaping the TiO2 into interdigitated electrodes (IDEs). The IDEs are treated with a plasma polymerization process to coat them with a thin film of poly(3,4-ethylenedioxythiophene) (PEDOT), which acts as a conductive layer to facilitate electron transfer.

With the conductive layer in place, the biosensor's surface is chemically activated to covalently bind the enzyme glucose oxidase (GOx), which is responsible for the specific recognition of glucose. The immobilization process involves activating the PEDOT film with a mixture of carbodiimide and N-hydroxysuccinimide, creating reactive sites for the GOx enzyme to attach. The GOx is then pipetted onto the surface and left to bind under controlled temperature and humidity to ensure maximum activity and stability of the enzyme.

The glucose biosensor incorporates a microfluidic channel system made from polydimethylsiloxane (PDMS), bonded to the silicon substrate using oxygen plasma treatment. This microfluidic integration allows for precise sample delivery to the sensor surface and removal of unreacted substances, ensuring a clear signal for glucose detection. The microfluidic system includes a sample inlet, reaction chamber, and waste outlet, facilitating continuous flow analysis.

Upon introduction of a glucose-containing sample to the microfluidic chamber, the GOx catalyzes the oxidation of glucose, producing gluconic acid and hydrogen peroxide. The generated hydrogen peroxide is then electrochemically reduced at the biosensor's electrode, producing a current proportional to the glucose concentration. This current is measured by an onboard potentiostat circuit, which is calibrated to translate electrical signals into precise glucose concentration readings.

The completed biosensor is housed within a biocompatible casing with an integrated glucose calibration system, enabling users to perform on-the-spot calibration for accurate readings. This biosensor model demonstrates a significant advancement in non-invasive glucose monitoring technology, offering rapid, precise measurements essential for effective diabetes management.

1. A biosensor comprising a three-dimensional structural core that enhances mechanical stability and electrical conductivity, thereby increasing the effective surface area for biomolecule detection relative to the physical dimensions of the biosensor.

2. The biosensor of claim 1, wherein the structural core is composed of various materials selected from a group consisting of metals, semiconductors, organic compounds, ceramics, and glass, fashioned into configurations that maximize surface area, including but not limited to columns and helices.

3. The biosensor of claim 2, wherein the structural core is overlaid with a selective layer for specific analyte detection, with the application of the selective layer being specified outside the scope of the current claims.

4. The biosensor of claim 1, wherein the structural core is fabricated using subtractive manufacturing techniques, incorporating the use of biodegradable materials for disposable sensors that maintain functionality for a predetermined lifespan before safe decomposition.

5. The biosensor of claim 1, wherein the structural core is produced via additive manufacturing methods, including the creation of a temporary mold followed by the addition of core material through deposition techniques, and possibly assembled on a flat substrate followed by pick-and-place and bonding techniques.

6. The biosensor of claim 1, further comprising a conductive layer applied by standard deposition techniques, with selective removal of this layer to manage electrical pathways, using processes such as sputter etching, ion milling, lift-off, and lithographic methods combined with chemical dissolution.

7. The biosensor of claim 1, further integrating microfluidic channels within the structural core to direct sample flow across the sensor surface, enhancing analyte-receptor interaction and improving sensitivity of detection.

8. The biosensor of claim 1, incorporating a thermal management system utilizing thermally conductive materials within the structural core to function as heat sinks, maintaining operational temperature for consistent sensor performance.

9. The biosensor of claim 8, wherein the thermal management system comprises a network of thermally conductive pathways made from materials like graphene or silver nanoparticles, to facilitate heat transfer and prevent localized overheating.

10. The biosensor of claim 8, employing a layered construction with alternating thermally conductive and insulating materials to manage heat flow in high-power operations or varying environmental temperatures.

11. The biosensor of claim 8, including a passive thermal conduction system comprising metal microfins or microchannels within the core to enhance cooling by natural convection.

12. The biosensor of claim 8, utilizing electrically insulating but thermally conductive adhesives for component assembly, enabling heat conduction without electrical interference.

13. The biosensor of claim 1, further including a copper-based thermal regulation layer featuring strategically placed through-holes, which not only contribute to the structural integrity but also enhance the sensor's thermal management capabilities, ensuring precise temperature control essential for the stability and accuracy of the biomolecular detection processes.

14. The biosensor of claim 1, designed to accommodate thermal expansion and contraction without loss of sensor integrity, by using materials with matching coefficients of thermal expansion or joints and interfaces that permit thermal movement.
